(19) United States
(12) Patent Application Publication
(10) Pub. No.: US 2021/0007854 A1
(43) Pub. Date: Jan. 14, 2021

(54) Complete Replacement Elbow Joint with Replaceable Parts

(71) Applicant: James Gordon Johnson, Webster City, IA (US)

(72) Inventor: James Gordon Johnson, Webster City, IA (US)

(21) Appl. No.: 16/501,944

(22) Filed: Jul. 10, 2019

(51) Int. Cl. A61F 2/38

(52) U.S. Cl. CPC A61F 2/3804; A61F 2002/3822; A61F 2002/3831; A61F 2002/3813

(57) ABSTRACT

This is an application for a patent filing of an original type of articulating prosthetic human elbow joints, with replaceable parts.

Intramedullary nails located on the base of each joint is used to secure the joint in bones of the humerus and ulna. The intramedullary nail has small pimples in it like a golf ball that allows bone cells to granulate in and attach themselves permanently to the intramedullary nail. This configuration requires only a small amount of adhesive for fixation until the bone cells granulate into the intramedullary nail for a firm permanent attachment.

The joint is equipped with humeral and ulnar pieces that contain the articulating surfaces for the joint. The articulating surfaces are replaceable, this and designed to take the bulk of the load. This is done so that the articulating services will break instead of the bones fracturing when too much of a load is applied to the prosthetic. This is done to alleviate the necessity of total replacement of the joint and allow repairs in situ in a quick and less invasive manner, which should reduce recovery time and time in physical therapy.

This is an unconstrained elbow joint

It depends on assistance from the synovial joint capsule and ligaments for proper support

There is a piece of spring steel on the lower portion that fits into the trochlear notch of the ulna that facilitates movement of the joint with little bone wear.

The joint is made to rotate to aide in supination and pronation of the joint and give a more normal feel of joint movement in everyday use.

This is accomplished with a bushing and a pin in the lower portion of the of the joint. The bushing and pin allow the radius and ulna which gives a more normal feel of supination and pronation.

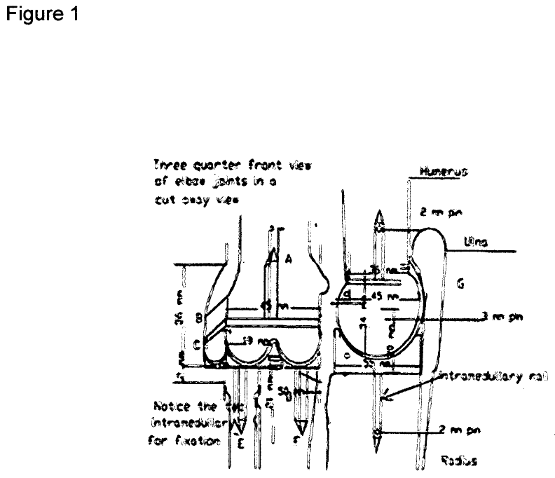

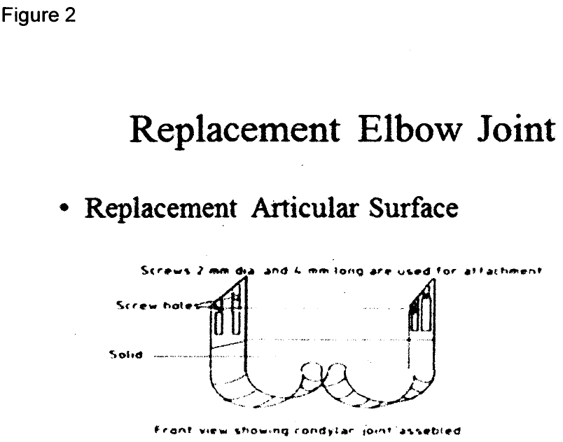

See FIG. 1.

If the joint articular surface becomes damaged, the damaged part is removed by the removal of four set screws two on each side. Then the damaged part is slid forward and the new one slid into place. Then secured by four set screws.

Elbow Joint Part

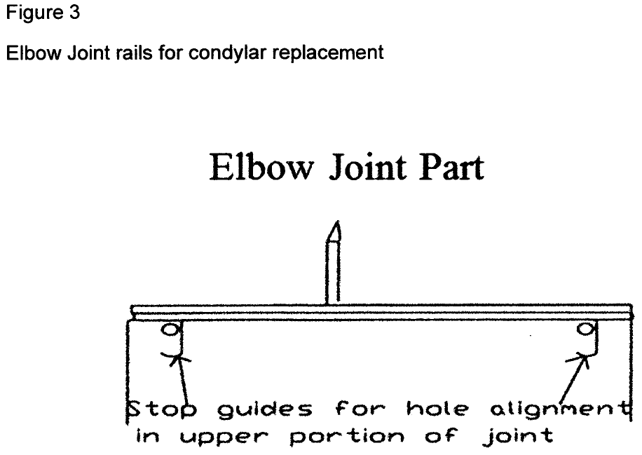

FIG. 3

This piece is the replacement part if the condylar part of the joints becomes broken

There are tracks on both side of the joint attach piece that allows for the broken part to be removed and replaced with a new condylar part

The part in (see FIG. 3) then slides into place and is secured by four set screws with counter sunk heads in the holes provided

This type of joint eliminates the necessity for complete joint replacement reducing trauma on the patient and a shorter physical therapy time.

1. A unconstrained replacement elbow joint with replaceable articular parts, containing:
the body of the joint with cupped intramedullary nail attach points for permanent attachment very little bone cement is needed;
intramedullary nail attach points with pimples on them like a golf ball that allow bone granulation to make attachment permanent;
unconstrained articular surfaces which move freely through the joints range of motion;
the distal portion of the prosthetic contains a spring steel piece that fits in the trochlear notch for smoother joint movement;
The distal portion of the joint is in two parts that rotate around a bushing in the middle for better range of motion;
articulating surfaces designed to break under severe load so that there is no breaking or cracking of the bone at the intramedullary nail attach points;
articulating surfaces that attach to the body of the joint on two rails, with stop guides that are molded on both sides of the upper and lower prosthetic;
the part is secured by two set screws on each side of the joint in holes that are provided in the permanently attached part of the joint;
the stop guides in the track let the surgeon know that the new articular portion is seated properly;
articulating surfaces attached to the rails by sunken head set screws;

2. A unconstrained elbow joint with replaceable articular parts according to claim 1, where the unconstrained joint with articular surfaces that break under extreme load;
replaces a patient's original joint with a prosthetic joint that contains replacement articular surfaces;
replacement articular surfaces have the benefits of a less invasive, quicker surgery and less time for the patient in PT;
Replacement articular surfaces are also a benefit for replacement when the surfaces get worn;
